(19) United States
(12) Patent Application Publication
(10) Pub. No.: US 2013/0172627 A1
(43) Pub. Date: Jul. 4, 2013

(54) PROCESS FOR PREPARING LOWER OLEFINS

(71) Applicant: Shell Oil Company, Houston, TX (US)

(72) Inventors: Leslie Andrew CHEWTER, Amsterdam (NL); Rajaram RAMESH, Amsterdam (NL); Jeroen VAN WESTRENEN, Amsterdam (NL)

(73) Assignee: SHELL OIL COMPANY, Houston, TX (US)

(21) Appl. No.: 13/727,863

(22) Filed: Dec. 27, 2012

(30) Foreign Application Priority Data
Dec. 28, 2011 (EP) 11195852.6

(51) Int. Cl. C07C 1/22; C07C 41/06; C07C 4/04

(52) U.S. Cl. CPC C07C 1/22; C07C 4/04; C07C 41/06
USPC 568/697; 585/302; 585/303

(57) ABSTRACT

The invention is a process for preparing lower olefins comprising: a) steam cracking a paraffinic feedstock to obtain a cracker effluent comprising olefins and saturated and unsaturated C4 hydrocarbons; b) contacting an oxygenate feedstock with a molecular sieve-comprising catalyst, at a temperature in the range of from 350 to 1000° C. to obtain an oxygenate conversion effluent comprising olefins and saturated and unsaturated C4 hydrocarbons; c) subjecting the cracker effluent and the oxygenate conversion effluent to one or more separation steps such that an olefin product stream comprising ethylene and/or propylene, and a stream comprising saturated and unsaturated C4 hydrocarbons are obtained; and d) subjecting part of the stream comprising C4 hydrocarbons from both the cracker effluent and the oxygenate conversion effluent to extractive distillation to obtain a stream enriched in unsaturated C4 hydrocarbons and a stream enriched in saturated C4 hydrocarbons.

This application claims the benefit of European Patent Application No. 11195852.6, filed Dec. 28, 2011, the entire disclosure of which is hereby incorporated by reference.

### FIELD OF THE INVENTION

The invention relates to a process for preparing lower olefins.

### BACKGROUND TO THE INVENTION

Conventionally, lower olefins such as ethylene and propylene are produced via steam cracking of hydrocarbon feedstocks including ethane, propane, naphtha, gasoil and hydrowax. An alternative route to lower olefins is the so-called oxygenate-to-olefin process. In such oxygenate-to-olefin process, an oxygenate such as methanol or dimethylether (DME) is provided to a reaction zone containing a suitable oxygenate conversion catalyst, typically a molecular sieve-comprising catalyst, and converted into ethylene and propylene.

Integrated processes for the manufacture of lower olefins that comprise both steam cracking of hydrocarbon feedstocks and an oxygenate-to-olefin process are known in the art. Such a process is for example disclosed in WO2011/057975.

In oxygenate-to-olefins conversion processes, not only the desired lower olefins are formed, since a substantial part of the oxygenate is converted into C4+ olefins and paraffins. In steam cracking, not all paraffinic feed is cracked into lower olefins. The effluent of a steam cracker may still contain unconverted paraffins. Moreover, C4+ olefins, including a substantial amount of di-olefins such as butadiene are formed. Thus, both the effluents of a steam cracker and of an oxygenate-to-olefin conversion process comprise substantial amounts of saturated and unsaturated C4+ hydrocarbons.

In the integrated process of WO2011/057975, the effluent of a steam cracker and of an oxygenate-to-olefins process are combined and fractionated in a common separator. A fraction comprising C4 hydrocarbons is obtained that is, after hydrogenation of the butadiene therein, recycled to the stream cracker and/or to the oxygenate-to-olefins process.

A disadvantage of the process of WO2011/057975 is that saturated C4 hydrocarbons that are present in the fraction comprising C4 hydrocarbons are recycled to the oxygenate-to-olefins process. Such saturated C4 hydrocarbons will not be converted to lower olefins in the oxygenate-to-olefins process. Moreover, in order to avoid undesired accumulation of paraffins in the recycle stream to the oxygenate-to-olefins process, the ratio between the amounts of C4 hydrocarbons recycled to the stream cracker and to the oxygenate-to-olefins process should be carefully controlled in the process of WO2011/057975. A further disadvantage of the process of WO2011/057975 process is that butadiene is hydrogenated and thus cannot be recovered as product.

### SUMMARY OF THE INVENTION

An improved process for the preparation of lower olefins that is integrating a steam cracker and an oxygenate-to-olefins process has been found. The improved process provides the possibility to selectively recycle saturated C4 hydrocarbons to the steam cracker and unsaturated C4 hydrocarbons to the oxygenate-to-olefins process and in some embodiments to export butadiene as a valuable product.

Accordingly, the present invention relates to a process for the preparing lower olefins, the process comprising the following steps:

a) steam cracking a paraffinic feedstock under cracking conditions in a cracking zone to obtain a cracker effluent comprising olefins and C4 hydrocarbons including saturated and unsaturated C4 hydrocarbons;
b) contacting an oxygenate feedstock with a molecular sieve-comprising catalyst, at a temperature in the range of from 350 to 1000° C. to obtain an oxygenate conversion effluent comprising olefins and C4 hydrocarbons including saturated and unsaturated C4 hydrocarbons;
c) subjecting the cracker effluent and the oxygenate conversion effluent to one or more separation steps such that at least an olefin product stream comprising ethylene and/or propylene, and a stream comprising C4 hydrocarbons including saturated and unsaturated C4 hydrocarbons from both the cracker effluent and the oxygenate conversion effluent, are obtained; and
d) subjecting at least part of the stream comprising C4 hydrocarbons from both the cracker effluent and the oxygenate conversion effluent to extractive distillation to obtain a stream enriched in unsaturated C4 hydrocarbons and a stream enriched in saturated C4 hydrocarbons.

An important advantage of the process according to the invention is that a stream enriched in unsaturated C4 hydrocarbons and a stream enriched in saturated hydrocarbons is obtained. Thus, it is possible to selectively recycle saturated hydrocarbons to steam cracker step a) and unsaturated C4 hydrocarbons to oxygenate-to-olefins step b).

### BRIEF DESCRIPTION OF THE DRAWINGS

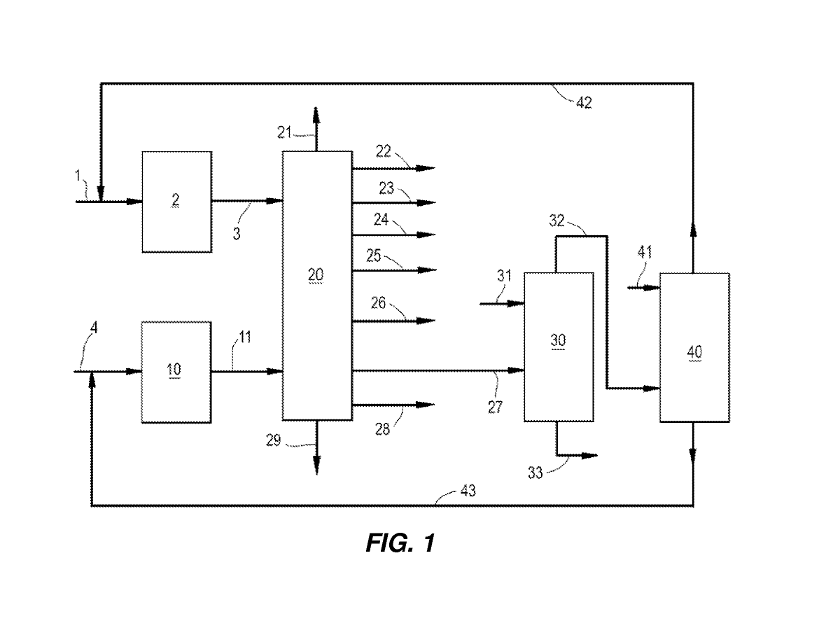

In each of FIGS. 1 to 3 is schematically shown a process according to the invention.

### DETAILED DESCRIPTION OF THE INVENTION

In the process according to the invention, a paraffinic feedstock is steam cracked under cracking conditions in a cracking zone to obtain a cracker effluent comprising olefins (step a)). An oxygenate is converted into lower olefins by contacting the oxygenate with a molecular sieve-comprising catalyst at a temperature in the range of from 350 to 1000° C. to obtain an oxygenate conversion effluent comprising olefins (step b)). In step c) the cracker effluent and the oxygenate conversion effluent are subjected to one or more separation steps to obtain at least an olefin product stream comprising ethylene and/or propylene, and a stream comprising C4 hydrocarbons from both the cracker effluent and the oxygenate conversion effluent. The stream comprising C4 hydrocarbons from both effluents, including saturated and unsaturated C4 hydrocarbons, is subjected to extractive distillation in step d) to obtain a stream enriched in unsaturated C4 hydrocarbons and a stream enriched in saturated C4 hydrocarbons.

In step a), a paraffinic feedstock is steam cracked in a cracking zone under cracking conditions to produce at least olefins and hydrogen. The cracking zone may comprise any cracking system known in that art that is suitable for cracking the paraffinic feedstock that is supplied to the cracking zone. The cracking zone may comprise one or more furnaces, each dedicated for a specific feed or fraction of the feed. The cracking system may for example be a naphtha cracker or an LPG cracker with a furnace equipped to process C3 and C4 paraffins and optionally a separate furnace for cracking ethane.

The paraffinic feedstock may be any suitable paraffinic feedstock. Preferably the paraffinic feedstock is a feedstock comprising light paraffins, i.e. C2-C5 paraffins, in particular C2-C4 paraffins, and/or naphtha. The feedstock may comprise non-paraffinic hydrocarbons such as olefins, preferably in quantities of less than 10 wt % based on the total weight of hydrocarbons. The paraffinic feed may comprise a recycle stream from the process.

The cracking step is performed at elevated temperatures, preferably in the range of from 650 to 1000° C., more preferably of from 680 to 830° C.

Steam is usually added to the cracking zone, acting as a diluent reducing the hydrocarbon partial pressure and thereby enhancing olefin yield. Steam also reduces the formation and deposition of carbonaceous material or coke in the cracking zone.

Steam cracking of paraffins is well known in the art. Reference is for instance made to Kniel et al., Ethylene, Keystone to the petrochemical industry, Marcel Dekker, Inc, New York, 1980, in particular chapter 6 and 7, as well as to US2005/0038304, WO2009/039948.

In addition to ethylene and propylene, other products are formed in steam cracking step a). Such products may include butylene, butadiene, ethyne, propyne, propadiene and benzene. Coke may also be formed and may require regular cleaning of the steam cracker furnace such as through decoking with air.

In oxygenate conversion step b), an oxygenate is converted into lower olefins by contacting the oxygenate with a molecular sieve-comprising catalyst at a temperature in the range of from 350 to 1000° C. Besides water and lower olefins, i.e. ethylene and propylene, C4 olefinic and paraffinic hydrocarbons and, in a lesser amount, C5+ olefinic and paraffinic hydrocarbons are formed as by-product. Small amounts of dienes like butadienes may be formed in step b).

Reference herein to an oxygenate is to a compound comprising at least one alkyl group that is covalently linked to an oxygen atom. Preferably, at least one alkyl group has up to five carbon atoms, more preferably up to four, even more preferably one or two carbon atoms, most preferably at least one alkyl group is methyl. Mono-alcohols and dialkylethers are particularly suitable oxygenates. Methanol and dimethylether or mixtures thereof are examples of particularly preferred oxygenates.

The oxygenate conversion in step b) is carried out by contacting the oxygenate with a molecular sieve-comprising catalyst at a temperature in the range of from 350 to 1000° C., preferably of from 350 to 750° C., more preferably of from 450 to 700° C., even more preferably of from 500 to 650° C. The conversion may be carried out at any suitable pressure, preferably at a pressure in the range of from 1 bar to 50 bar (absolute), more preferably of from 1 bar to 15 bar (absolute). A pressure in the range of from 1.5 to 4.0 bar (absolute) is particularly preferred.

Any molecular sieve comprising catalyst known to be suitable for the conversion of oxygenates, in particular alkanols and dialkylethers, into lower olefins may be used. Preferably the catalyst comprises a molecular sieve having a 8-, 10- or 12-ring structure and an average pore size in the range of from 3 Å to 15 Å. Examples of suitable molecular sieves are silicoaluminophosphates (SAPOs), aluminophosphates (AlPO), metal-substituted aluminophosphates or metal-substituted silicoaluminophosphates. Preferred SAPOs include SAPO-5, -8, -11, -17, -18, -20, -31, -34, -35, -36, -37, -40, -41, -42, -44, -47 and -56. SAPO-17, -18, -34, -35, and -44 are particularly preferred.

A particular suitable class of molecular sieves are zeolites. In particular in case not only oxygenates but also C4+ olefins or compounds that form C4+ olefins under the reaction conditions prevailing in oxygenate conversion step b) are supplied to step b), e.g. a tertiary alkylether such as methyl tertiary butylether, a zeolite-comprising catalyst is preferred as molecular-sieve comprising catalyst, more preferably a catalyst comprising a zeolite with at least a 10-membered ring structure. Zeolite-comprising catalysts are known for their ability to convert higher olefins to lower olefins, in particular C4+ olefins to ethylene and/or propylene. Suitable zeolite-comprising catalysts include those containing a zeolite of the ZSM group, in particular of the MFI type, such as ZSM-5, the MTT type, such as ZSM-23, the TON type, such as ZSM-22, the MEL type, such as ZSM-11, the FER type. Other suitable zeolites are for example zeolites of the STF-type, such as SSZ-35, the SFF type, such as SSZ-44 and the EU-2 type, such as ZSM-48. Preferably, the catalyst comprises at least one zeolite selected from MFI, MEL, TON and MTT type zeolites, more preferably at least one of ZSM-5, ZSM-11, ZSM-22 and ZSM-23 zeolites.

The zeolite in the oxygenate conversion catalyst is preferably predominantly in the hydrogen form. Preferably at least 50 wt %, more preferably at least 80 wt %, even more preferably at least 95 wt %, still more preferably at least 100 wt % of the zeolite is in the hydrogen form.

The molecular sieve-comprising catalyst may further comprise a binder material such as for example silica, alumina, silica-alumina, titania, or zirconia, a matrix material such as for example a clay, and/or a filler.

The present molecular sieve catalyst may comprise phosphorus as such or in a compound, i.e. phosphorous other than any phosphorus included in the framework of the molecular sieve. It is preferred that an MEL or MFI-type zeolite comprising catalyst additionally comprises phosphorus. The phosphorus may be introduced by pre-treating the MEL or MFI-type zeolites prior to formulating the catalyst and/or by post-treating the formulated catalyst comprising the MEL or MFI-type zeolites. Preferably, the present molecular sieve catalyst comprising MEL or MFI-type zeolites comprises phosphorus as such or in a compound in an elemental amount of from 0.05-10 wt % based on the weight of the formulated catalyst. A particularly preferred catalyst comprises phosphorus and MEL or MFI-type zeolites having SAR of in the range of from 60 to 150, more preferably of from 80 to 100. An even more particularly preferred catalyst comprises phosphorus and ZSM-5 having SAR of in the range of from 60 to 150, more preferably of from 80 to 100.

In step c), the cracker effluent and the oxygenate conversion effluent to one or more separation steps such that at least an olefin product stream comprising ethylene and/or propylene, and a stream comprising C4 hydrocarbons including saturated and unsaturated C4 hydrocarbons from both the cracker effluent and the oxygenate conversion effluent, are obtained. Step c) may be carried out by combining the cracker effluent and the oxygenate conversion effluent to obtain a combined effluent and subjecting the combined effluent to one or more separation steps to obtain at least an olefin product stream comprising ethylene and/or propylene from both effluents and a stream comprising C4 hydrocarbons from both effluents. Alternatively, each of the effluents is separately subjecting to one or more separation steps such that a first stream comprising C4 hydrocarbons and a second stream comprising C4 hydrocarbons are obtained, the first stream comprising C4 hydrocarbons from the cracker effluent and the second stream comprising C4 hydrocarbons from the oxygenate conversion effluent. The first and the second stream are then combined to obtain the stream comprising C4 hydrocarbons, including saturated and unsaturated C4 hydrocarbons, from both the cracker effluent and the oxygenate conversion effluent.

The stream comprising C4 hydrocarbons from both the cracker effluent and the oxygenate conversion effluent may comprise heavier hydrocarbons such as C5 and/or C6 hydrocarbons.

Typically, both the steam cracker effluent and the oxygenate conversion effluent will contain C4 hydrocarbons, in particular unsaturated C4 hydrocarbons. The cracker effluent will typically contain more butadiene than the oxygenate conversion effluent, in particular in case the paraffinic feedstock comprises C4 hydrocarbons.

Effluents from cracking and oxygenate conversion need to be worked up in order to separate and purify various desired components, and in particular to separate one or more lower olefins product streams. Typically, in known steam cracking as well as in oxygenate-to olefins processes, the effluent is quenched in a quench unit with quench medium such as water, in order to cool the gaseous effluent to a temperature close to ambient temperature before it is fed to a compressor. Any liquid heavy hydrocarbons are phase separated from liquid water and separately recovered. Water or steam from the quench unit may be partially recycled as diluent to cracking step a) and/or to oxygenate conversion step b), optionally after treatment or purification, e.g. to remove catalyst fines. Vapour components after quench are typically sent to a compression section, subjected to a caustic wash treatment, dried, and sent to a separation system including a cold section, so as to obtain separate streams of main components, such as for example hydrogen, light ends typically including methane and/or CO, ethane, ethylene, propane, propylene, a C4 fraction, a C5+ fraction, and water. There may also be a separate outlet for heavy (liquid) hydrocarbons. It will be understood that the separation may be conducted differently, such that certain streams are combined, or further separation may be carried out.

It will be understood, and this is for example discussed in US2005/0038304, that cracker and oxygenate conversion effluents may be combined at various stages of work-up, such as before quenching, after quenching, before compression, or after compression. Even if the effluents are combined before quenching, certain process steps such as cooling/heat exchanging can be carried out on one or both effluents separately. In case C4 or heavier hydrocarbons are fed to steam cracking step a), a primary fractionation unit might be needed to remove a pyrolysis gasoline fraction from the cracker effluent before it is further quenched in a quench tower. In case a sufficiently light paraffinic feed is fed to the cracking zone, it is typically not required to include a primary fractionator for separating heavy components from the cracker effluent before quenching.

In step d), at least part of the stream comprising C4 hydrocarbons from both the cracker effluent and the oxygenate conversion effluent obtained in step c) is subjected to extractive distillation to obtain a stream enriched in unsaturated C4 hydrocarbons and a stream enriched in saturated C4 hydrocarbons. Preferably, the stream subjected to extractive distillation in step d) contains no or only a low amount of butadiene. Therefore, butadiene, which is typically formed in step a) in case a feed comprising C4 hydrocarbons is used, is preferably removed from the C4 hydrocarbon stream prior to subjecting such stream to step d). In case the effluents are combined prior to separating a stream comprising C4 hydrocarbons from the combined effluent, butadiene is preferably removed from the stream comprising C4 hydrocarbons from both effluents. In case the effluents are each subjected to one or more separate separation steps and a first and a second stream comprising C4 hydrocarbons from the cracker effluent and the oxygenate conversion effluent, respectively, are obtained, butadiene is preferably removed from the first stream, i.e. the stream comprising C4 hydrocarbons from the cracker effluent.

Butadiene may be removed by selectively hydrogenating the butadiene by contacting the relevant C4 hydrocarbon stream with a hydrogenation catalyst in the presence of hydrogen under conditions such that butadiene is selectively hydrogenated to butene. The hydrogenation of butene to butane is preferably minimized. Suitable processes for selective hydrogenation of butadiene are well-known in the art (also referred to as partial, mild or semi-hydrogenation). Any suitable conditions known in the art may be applied. Reference is made by way of example to Derrien, M. L. “Selective hydrogenation applied to the refining of petrochemical raw materials produced by steam cracking” (1986) Stud. Surf. Sci. Catal., 27, pp. 613-666, to WO 95/15934, or to US4695560. Typically at least 90 wt % of butadiene is converted to butene, and less than 10 wt %, preferably less than 5 wt %, of the butene, based on butene in the feed to the selective hydrogenation, is converted to butane.

Alternatively and preferably, butadiene is be removed from the stream comprising C4 hydrocarbons as butadiene. An important advantage of removing butadiene as butadiene, is that butadiene may be obtained as valuable product from the process. Such removal of butadiene is preferably done by extractive distillation. If extractive distillation is used for butadiene removal, the process comprises two extractive distillation steps: a first extractive distillation step to remove butadiene from the stream comprising C4 hydrocarbons from both effluents or from the stream comprising C4 hydrocarbons from the cracker effluent and a second extractive distillation step (step d) in the process according to the invention) to separate butenes from butanes. In the first extractive distillation step, the relevant C4 hydrocarbon stream and a suitable solvent are supplied to a first extractive distillation column. A butadiene-depleted C4 hydrocarbon stream comprising butenes and butanes will leave the column over the top and solvent containing butadiene leaves the column via the bottom. Butadiene may be obtained as product by stripping the solvent from the bottom stream.

In extractive distillation step d) at least part of the stream comprising C4 hydrocarbons from both effluents is supplied, together with a suitable solvent, to an extractive distillation column. Examples of suitable solvents are dimethylformamide (DMF), N-formylmorpholine (NFM), acetonitrile and N-methylpyrrolidone. Saturated C4 hydrocarbons will leave the column over the top and solvent containing unsaturated C4 hydrocarbons will leave the column via the bottom. In case the stream comprising C4 hydrocarbons from both effluents comprises C5+ hydrocarbons, the bottom fraction will further comprise such C5+ hydrocarbons.

Separation of butanes from butenes by means of extractive distillation is well-known in the art. Any suitable process conditions and solvents known in the art may be applied.

Solvent is separated from the butenes by means known in the art, typically by stripping, in order to obtain the stream enriched in unsaturated C4 hydrocarbons. In case the process comprises two extractive distillation steps, i.e. one for butadiene removal and one for butene/butane separation, the same solvent is preferably used in both extractive distillation steps.

The stream enriched in saturated C4 hydrocarbons (top stream of the extractive distillation column) may be withdrawn from the process, for example to be blended into an LPG pool or to be isomerised to isobutane. Preferably, at least part of the stream enriched in saturated C4 hydrocarbons is recycled to steam cracking step a).

At least part of the stream enriched in unsaturated C4 hydrocarbons (bottom stream) may be is recycled to oxygenate conversion step b).

Preferably, at least part of the stream enriched in unsaturated C4 hydrocarbons is subjected to an etherification step (step e)) for conversion of isobutene into an alkyl tertiary butyl ether. Thus, an etherification product stream comprising alkyl tertiary butyl ether is obtained. In a subsequent separation step f), the etherification product stream is then separated into an alkyl tertiary butyl-ether enriched stream and an isobutene-depleted unsaturated C4 hydrocarbon stream. At least part of the isobutene-depleted unsaturated C4 hydrocarbon stream and/or at least part of the alkyl tertiary butyl ether is then recycled to oxygenate conversion step b).

In etherification step e), at least part of the stream enriched in unsaturated C4 hydrocarbons is supplied to an etherification reaction zone together with an alcohol. The etherification zone comprises an etherification catalyst and in this zone at least part of the isobutene in the stream enriched in unsaturated C4 hydrocarbons is reacted with the alcohol to be converted into alkyl tertiary butyl ether. In case the stream enriched in unsaturated C4 hydrocarbons also comprises iso-pentenes, also alkyl tertiary amyl ether is formed. Thus, an etherification product stream comprising alkyl tertiary butyl ether is obtained. The alcohol is selected from the group consisting of methanol, ethanol and a mixture thereof. Preferably, the alcohol is methanol and an etherification product stream comprising methyl tertiary butyl ether is obtained. Etherification of isobutene to form an alkyl tertiary butyl ether is well-known in the art. Any catalyst and process conditions known to be suitable for such etherification may be used. Typically, the etherification catalyst is an acid catalyst. Preferably, the etherification catalyst is a protonated cation-exchange resin or a heteropolyacid promoted by a metal. A particularly preferred catalyst is Amberlyst-15. Preferably, the etherification reaction is carried out at a temperature in the range of from 40 to 100° C., more preferably of from 50 to 85° C. The reaction may be carried out at any suitable pressure, preferably in the range of from 1 to 20 bar (absolute), more preferably of from 5 to 15 bar (absolute).

In a subsequent step f), the etherification product stream is separated into an alkyl tertiary butyl ether-enriched stream and an isobutene-depleted unsaturated C4 hydrocarbon stream. This may be done by any suitable means known in the art, for example by distillation.

In step g), at least part of the isobutene-depleted unsaturated C4 hydrocarbon stream and/or at least part of the alkyl tertiary-butyl ether is recycled to oxygenate-to-olefin conversion step b).

Preferably, at least part of the isobutene-depleted unsaturated C4 hydrocarbon stream is recycled to step b), more preferably at least 50 wt %, even more preferably at least 90 wt %. Since at least the amount of saturated hydrocarbons produced per unit of time in step b) is removed from the C4 hydrocarbon stream in step d), the entire isobutene-depleted unsaturated C4 hydrocarbon stream may be recycled to step b). Recycling of a stream depleted in isobutene is particularly advantageous in case a molecular sieve comprising catalyst is used in step a) that does not or does hardly catalyse the conversion of isobutene into lower olefins. Examples of such catalysts are SAPO-containing catalysts, in particular a SAPO-34 containing catalyst.

Alternatively, or in combination with a recycle of at least part of the isobutene-depleted unsaturated C4 hydrocarbon stream, at least part of the alkyl tertiary-butyl ether produced in step e) is recycled to oxygenate-to-olefin conversion step b). Such recycle of alkyl tertiary-butyl ether is advantageous in case a molecular sieve comprising catalyst is used in step b) that is able to catalyse the conversion of isobutene into lower olefins, as is typically the case for a zeolite-comprising catalyst, in particular a catalyst comprising a zeolite with a 10-membered ring structure. Alkyl tertiary-butyl ether may be recycled as such to step b) or in the form of tertiary butanol and/or isobutene, i.e. after conversion into tertiary butanol and/or isobutene.

The alkyl tertiary-butyl ether may be decomposed to back to the alcohol and the iso-olefins, or optionally the alcohol and an iso-paraffin. In that case the methanol may be recycled to the etherification process.

The alkyl tertiary butyl ether formed in step e) can advantageously be converted into tertiary butyl hydroperoxide, which can be converted into an epoxide by reacting it with ethylene and/or propylene obtained in step c). Thus, an integrated process for preparing an epoxide from an oxygenate is provided. Such process further comprises: converting at least part of the alkyl tertiary butyl ether in the alkyl tertiary butyl ether-enriched stream into the alcohol and isobutane (step h)); oxidizing isobutane obtained in step h) into tertiary butyl hydroperoxide (step i)); and reacting the tertiary butyl hydroperoxide with ethylene and/or propylene separated from the olefinic product stream obtained in step c) to obtain the epoxide and tertiary butanol (step j)).

At least part of the alkyl tertiary butyl ether in the alkyl tertiary butyl ether-enriched stream may converted into the alcohol and isobutane, in an optional step (h). For example by first cracking alkyl tertiary butyl ether into isobutene and the alcohol and then hydrogenating the isobutene thus-formed into isobutane. The cracking of a tertiary alkyl ether into its corresponding alcohol and iso-olefin and the hydrogenation of an iso-olefin into its corresponding iso-alkane are well-known in the art. Alternatively, the tertiary alkyl ether can be hydrocracked directly into its corresponding alcohol and iso-paraffin in a single step. The cracking and hydrogenation may be carried out in any suitable way known in the art. In the cracking step, preferably an acid catalyst is used. Preferred cracking catalysts include acid cation-exchange resins, heteropolyacids, metal oxides such as for example alumina or silica-alumina.

The cracking is preferably carried out at a temperature in the range of from 100 to 250° C., more preferably of from 120 to 200° C. The pressure is preferably in the range of from 1 to 10 bar (absolute).

Alternatively and preferably, the alkyl tertiary butyl ether is directly converted into tertiary butane and the alcohol, i.e. in a single step. The cracking and hydrogenation is then combined by contacting the alkyl tertiary butyl ether with a hydrocracking catalyst in the presence of hydrogen. Any suitable hydrocracking catalyst may be used for this step. Such catalyst comprises a hydrogenating function, preferably a hydrogenating metal, supported on an acidic support material. Preferably, the catalyst comprises an acidic support material selected from zeolitic or amorphous silica alumina and alumina. Amorphous silica alumina is a particularly preferred support material. The hydrogenation function is preferably a hydrogenating metal selected from Group VIII metals, more preferably selected from Pt, Pd, Ru, Rh, Ir, Ni and combinations thereof. Hydrogenating metal that do not easily convert methanol into carbon monoxide and hydrogen under the hydrocracking conditions prevailing in this step are particularly preferred. Examples of such hydrogenating metals are Pt and a combination of Pt and Ru.

Hydrocracking is preferably carried out at a temperature of at most 200° C. A higher temperature will result in a larger amounts of undesired by-products such as isobutene and dialkyl ether. More preferably, the temperature is in the range of from 50 to 200° C., even more preferably of from 60 to 180° C., still more preferably of from 80 to 150° C. A temperature in the range of from 85 to 120° C. is particularly preferred. Preferably, the pressure in step h) is such that the alkyl tertiary butyl ether is predominantly, i.e. at least 80 wt %, preferably at least 90 wt %, in the liquid phase. Preferably, the total pressure in step h) is in the range of from 1 to 35 bar (absolute).

The alcohol obtained in step h) is preferably recycled to oxygenate conversion step b) and/or to etherification step e).

In peroxidation step i), the isobutane obtained in step h) is oxidized into tertiary butyl hydroperoxide. Such peroxidation step is well-known in the art.

In step j), the tertiary butyl hydroperoxide obtained in step i) is reacted with ethylene and/or propylene separated in step c) to obtain the epoxide and tertiary butanol. Such epoxidation step is well-known in the art. Preferably, the tertiary butyl hydroperoxide is reacted with propylene to obtain propylene oxide.

In case propylene oxide is obtained in step j), the process according to the invention preferably further comprises converting the propylene oxide obtained into one or more polyether polyols, propylene glycol or propylene glycol ethers. Such conversion is known in the art and any suitable process conditions known in the art may be used. Polyether polyols can suitably be reacted with isocyanate to manufacture polyurethane.

The tertiary butanol obtained in step j) is preferably kept in the process by recycling it to step b), and/or, after dehydration to isobutene, to alkyl tertiary butyl ether conversion step e). Under the reaction conditions prevailing in step b), tertiary butanol will be dehydrated and water and isobutene are formed. If the catalyst in step b) is able to catalyse conversion of isobutene into lower olefins, part of the isobutene thus-formed will be further converted in lower olefins in step b). In order to maximise the propylene oxide yield of the process, it is, however, advantageous to keep a large part of the tertiary butanol formed in the process as an iso-C4 compound that can easily be converted into isobutane. Such isobutane can then be peroxidised to the tertiary butyl hydroperoxide that is needed for propylene oxide production in step j). Therefore, if the catalyst in step b) is able to convert isobutene into lower olefins, it is preferred to recycle at least part of the tertiary butanol formed in step j), after dehydration of the tertiary butanol to isobutene, as isobutene to alkyl tertiary butyl ether conversion step h). More preferably, part of the butanol formed in step j) is recycled to step b) and part of the tertiary butanol is recycled to step h). If recycled to both steps b) and h), the tertiary butanol may be recycled to both steps in the form of isobutene, i.e. after dehydration of the tertiary butanol. Alternatively, it is recycled as tertiary butanol to step b) and as isobutene to step h). The isobutene recycled to step h) will be hydrogenated to isobutane in step h). It will be appreciated that if step h) comprises separate cracking and hydrogenating steps, the isobutene will be recycled directly to the hydrogenating step.

Dehydration of tertiary butanol to isobutene is well-known in the art. The dehydration of tertiary butanol may be carried out using catalysts and process conditions known in the art.

Also in oxidation step i) tertiary butanol is produced as by-product. The tertiary butanol obtained in step i) may be recycled to step b) and/or step h) together with the tertiary butanol from step j).

Where the olefin product stream in step (c) comprises ethylene, least part of the ethylene may be further converted into at least one of polyethylene, mono-ethylene-glycol, ethylbenzene and styrene monomer. Where the olefin product stream in step (c) comprises propylene, at least part of the propylene may be further converted into at least one of polypropylene and propylene oxide.

### DETAILED DESCRIPTION OF THE DRAWINGS

In FIG. 1, an embodiment of the invention is schematically shown wherein the cracker effluent and the oxygenate conversion effluent are combined to form a combined effluent and the combined effluent is subjected to separation. The stream enriched in saturated C4 hydrocarbons obtained in extractive distillation step d) is recycled to steam cracking step a) and the stream enriched in unsaturated C4 hydrocarbons obtained in extractive distillation step d) is recycled to oxygenate conversion step b).

A naphtha stream is fed via line 1 to cracking zone 2 wherein it is steam cracked to obtain a cracker effluent that is withdrawn from cracking zone 2 via line 3.

Methanol is fed via line 4 to oxygenate conversion reaction zone 10 comprising an oxygenate conversion catalyst. In reaction zone 10, methanol is converted into olefins and water. The effluents of both cracking zone 2 and oxygenate conversion reaction zone 10 are supplied via lines 3 and 11, respectively, to separation zone 20 wherein the combined effluent is separated into a stream of hydrogen 21, light ends 22, ethane 23, ethylene 24, propane 25, propylene 26, a fraction comprising C4 hydrocarbons 27, a fraction comprising C5+ hydrocarbons 28 and water 29.

The fraction comprising C4 hydrocarbons 27 is fed to first extractive distillation column 30. Solvent (NFM) is supplied to column 30 via line 31. A stream comprising solvent and butadiene is withdrawn from the bottom of column 30, and a stream comprising butanes and butanes is withdrawn from the top of column 30 via line 32 and supplied to second extractive distillation column 40. Traces of butadiene that might be present in the top stream of column 30 may be removed by selective hydrogenation prior to supplying the top stream to second extractive distillation column 40. In column 40, the stream comprising butenes and butanes is separated into a stream enriched in butanes and a stream enriched in butenes. To column 40, NFM is supplied as solvent via line 41. A stream enriched in butanes is withdrawn from the top of column 40 via line 42 and recycled to steam cracking zone 2. A stream comprising NFM and butenes is withdrawn from the bottom of column 40 via line 43. After removal of the NFM (not shown) a stream enriched in butenes is recycled to oxygenate conversion reaction zone 10.

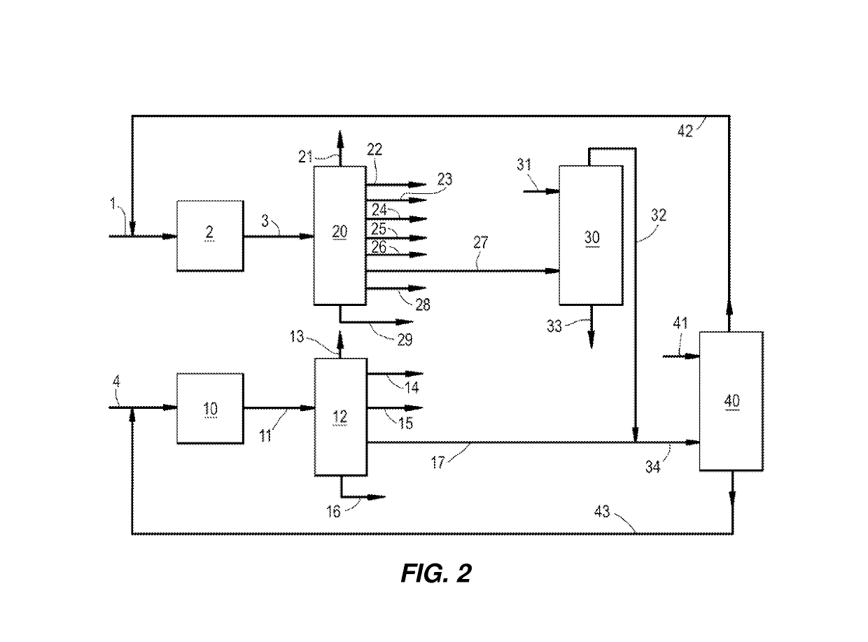

In FIG. 2, an embodiment of the invention is shown wherein the effluents of the cracker and of the oxygenate conversion each are separated in a separate separation zone and C4 hydrocarbon fractions from each effluent are combined prior to an extractive distillation step for butane/butane separation. Corresponding reference numbers have the same meaning as in FIG. 1.

The effluent of cracking zone 2 is supplied via line 3 to separation zone 20 wherein the cracker effluent is separated into a stream of hydrogen 21, light ends 22, ethane 23, ethylene 24, propane 25, propylene 26, a fraction comprising C4 hydrocarbons 27, a fraction comprising C5+ hydrocarbons 28 and water 29. The fraction comprising C4 hydrocarbons 27, i.e. the first stream comprising C4 hydrocarbons, is fed to first extractive distillation column 30 for butadiene removal. Solvent (NFM) is supplied to column 30 via line 31. A stream comprising solvent and butadiene is withdrawn from the bottom of column 30 via line 33, and a stream comprising butanes and butanes is withdrawn from the top of column 30 via line 32.

The effluent of oxygenate conversion zone 10 is supplied via line 11 to separation zone 12 wherein the effluent is separated into light ends comprising light by-products such as methane and carbon oxides, an ethylene-rich stream, a propylene-rich stream, a stream comprising C4 hydrocarbons and a fraction rich in C5+ hydrocarbons. The light ends, the ethylene-rich stream, the propylene-rich stream and the fraction rich in C5+ hydrocarbons are withdrawn from separation zone 12 via lines 13, 14, 15 and 16, respectively. The stream comprising C4 hydrocarbons, i.e. the second stream comprising C4 hydrocarbons, is withdrawn via line 17 and combined with the stream comprising butanes and butanes from distillation column 30. A combined stream comprising C4 hydrocarbons is then supplied to extractive distillation column 40 via line 34.

In column 40, the combined stream comprising C4 hydrocarbons is separated into a stream enriched in butanes and a stream enriched in butenes. To column 40, NFM is supplied as solvent via line 41. A stream enriched in butanes is withdrawn from the top of column 40 via line 42 and recycled to steam cracking zone 2. A stream comprising NFM and butenes is withdrawn from the bottom of column 40 via line 43. After removal of the NFM (not shown) a stream enriched in butenes is recycled to oxygenate conversion reaction zone 10.

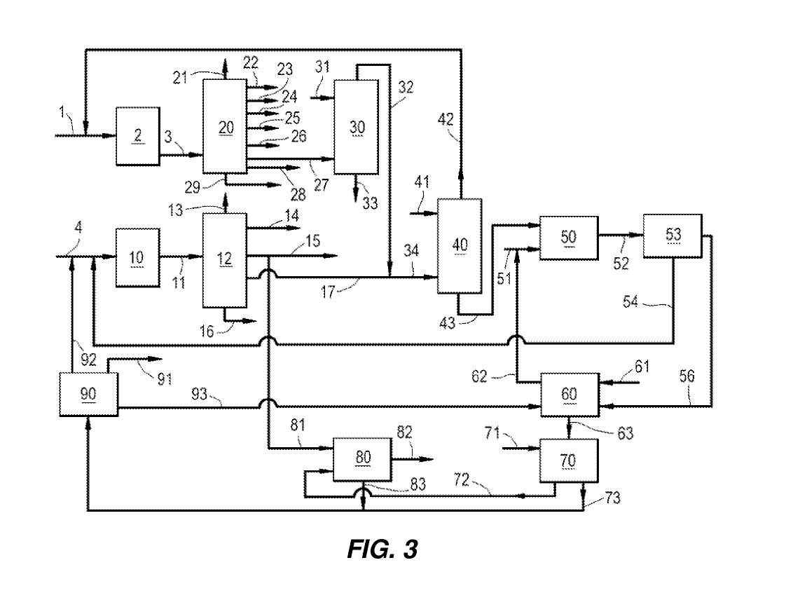

In FIG. 3, an embodiment of the invention is shown wherein the production of lower olefins is integrated with the manufacture of epoxide from lower olefins thus produced. Corresponding reference numbers have the same meaning as in FIG. 2. In the process as shown in FIG. 3, the stream enriched in butenes that is obtained after solvent removal from the bottoms of second extractive distillation column 40 is via line 43 supplied to etherification reaction zone 50. Methanol is supplied via line 51 to reaction zone 50 comprising an etherification catalyst. In etherification reaction zone 50, isobutene is reacted with methanol to form methyl tert-butyl ether (MtBE). The effluent of reaction zone 50 is supplied via line 52 to separator 53 to be separated into an isobutene depleted C4 hydrocarbon stream and an MtBE-enriched stream. The isobutene depleted C4 hydrocarbon stream is recycled to reaction zone 10 via line 54. The MtBE-enriched stream is withdrawn from separator 53 via line 56 and supplied to MtBE hydrocracking zone 60. Hydrogen is supplied to hydrocracking zone 60 via line 61. In zone 60, MtBE is converted into methanol and isobutane. Methanol is recycled to etherification zone 50 via line 62. Part of the methanol may be recycled to oxygenate conversion zone 10 (recycle not shown). Isobutane obtained in zone 60 is supplied via line 63 to oxidation reaction zone 70. Air is supplied as oxidant to zone 70 via line 71. In zone 70, isobutane is oxidised to tertiary butyl hydroperoxide and tertiary butanol. The tertiary butyl hydroperoxide formed in zone 70 is supplied via line 72 to epoxidation zone 80. The tertiary butanol formed is withdrawn via line 73. Part of the propylene-rich stream separated in fractionation section 20 is supplied to zone 80 via line 81. In zone 80, propylene oxide and tertiary butanol are formed. Propylene oxide is withdrawn as product via line 82. The tertiary butanol formed is withdrawn via line 83 and, combined with the tertiary butanol in line 73, supplied to tertiary butanol dehydration zone 90 and dehydrated into isobutene. Water is withdrawn from zone 90 via line 91. Part of the isobutene thus-formed is recycled to oxygenate conversion zone 10 via line 92 and part of the isobutene is recycled to MtBE hydrocracking zone 60 via line 93.

The invention is illustrated by the following non-limiting examples.

### EXAMPLES

Model calculations were carried out for a process configuration as shown in FIG. 3.

A stream of 3056 kilotons per annum (kton/a) of methanol, 172 kton/a of a recycle stream of isobutene and 299 kton/a of a recycle stream of isobutene-depleted C4 hydrocarbons including 12 kton/a methanol are supplied to oxygenate conversion zone 10. The isobutene-depleted C4 hydrocarbons stream comprises 246 kton/a of normal butenes, 53 kton/a of C4 paraffins and 12 kta of methanol. Zone 10 contains a zeolitic catalyst comprising ZSM-23 and ZSM-5 with a silica-to-alumina ratio of 280 in a weight ratio of 4 to 1, a binder and a matrix material. In zone 10, water and an olefinic product stream are formed. Fractionation in separation zone 12 yields 1146 kton/a of lower olefins, a C4 hydrocarbon fraction (363 kton/a) and a stream rich in C5+ hydrocarbons. A stream comprising C4 hydrocarbons from a naphtha stream cracker effluent, i.e. the stream in line 27, of 300 kton/a is fed to first extractive distillation zone 30 with acetonitril as extraction solvent. A C4 raffinate stream is obtained as top stream, which contains 14 kton/a of saturated C4 hydrocarbons, 70 kton/a of normal-butenes, and 59 kton/a of isobutene. The bottom stream contains 154 kton/a of butadiene and comprises the extractive solvent. The C4 raffinate stream is combined with the C4 hydrocarbon fraction from separation zone 12. The combined C4 hydrocarbon stream (505 kton/a) is fed to second extractive distillation zone 40.

In extractive distillation zone 40, the combined stream is contacted with NFM as solvent in a solvent to feed ratio of 5. A top product stream comprising 31 kton/a saturated C4 hydrocarbons and 3 kton/a butenes (both n-butenes and iso-butene) is obtained. This top stream is recycled to steam cracking zone 2. The 31 kton/a of saturated C4 hydrocarbons in the top stream from extractive distillation zone 40 match the amount of saturated C4 hydrocarbons produced in oxygenate-to-olefins reaction zone 10 plus the amount of C4 hydrocarbons in the C4 raffinate stream in line 32. Hence, saturated C4 hydrocarbons are not built up in the recycle to reaction zone 10.

A bottom product stream comprising solvent and unsaturated C4 hydrocarbons is obtained in extractive distillation zone 40. The NFM solvent is removed in a stripping zone (not shown). After stripping, a stream enriched in unsaturated C4 hydrocarbon is obtained that contains 248 kton/a of n-butenes, 174 kton/a of isobutene, 53 kton/a of saturated C4 hydrocarbons. This stream and 110 kton/a of methanol (98 kton/a recycled from MtBE hydrocracking zone 60 and 12 kton/a make-up from fresh methanol supply) is fed to etherification reaction zone 50 and MtBE is formed. After separation of the MtBE from the remaining C4 hydrocarbons, an azeotropic stream of 299 kton/a of isobutene-depleted C4 hydrocarbons and 12 kta of methanol is obtained and recycled to the reaction zone 10.

271 kton/a of MtBE, 310 kton/a of isobutene from isobutanol dehydration zone 90, and 17 kton/a of hydrogen are fed to MtBE hydrocracking zone 60 to form 500 kton/a of isobutane and 98 kton/a of methanol. The methanol is recycled to etherification reaction zone 50. The isobutane is supplied, together with air to oxidation reaction zone 70 to form tertiary butyl hydroperoxide and tertiary butanol. The tertiary butyl hydroperoxide and 181 kton/a of the propylene produced in zone 10 are converted into 250 kton/a of propylene oxide in epoxidation zone 80. The tertiary butanol formed in zones 70 and 80 is dehydrated in dehydration zone 90 to form 155 kton/a of water, and 482 kton/a of isobutene. Part of the isobutene (172 kton/a) is recycled to oxygenate conversion zone 10 and part (310 kton/a) is recycled to MtBE hydrocracking zone 60.

TABLE
Product streams in kilotons per annum in the EXAMPLE

| line | Compound | EXAMPLE |
|---|---|---|
| 1 | methanol | 3056 |
| 42 | Normal-butane and isobutane | 34 |
| 54 | isobutene-depleted C4 hydrocarbons | 311 |
| | including MeOH (azeotropic stream) | |
| 61 | hydrogen | 17 |
| 63 | isobutane | 500 |
| 81 | propylene | 181 |
| 82 | propylene oxide | 250 |
| 92 | isobutene | 172 |
| 93 | isobutene | 310 |

What is claimed is:

1. A process for preparing lower olefins, the process comprising the following steps:
a) steam cracking a paraffinic feedstock under cracking conditions in a cracking zone to obtain a cracker effluent comprising olefins and C4 hydrocarbons including saturated and unsaturated C4 hydrocarbons;
b) contacting an oxygenate feedstock with a molecular sieve-comprising catalyst, at a temperature in the range of from 350 to 1000° C. to obtain an oxygenate conversion effluent comprising olefins and C4 hydrocarbons including saturated and unsaturated C4 hydrocarbons;
c) subjecting the cracker effluent and the oxygenate conversion effluent to one or more separation steps such that at least an olefin product stream comprising ethylene and/or propylene, and a stream comprising C4 hydrocarbons including saturated and unsaturated C4 hydrocarbons from both the cracker effluent and the oxygenate conversion effluent, are obtained; and
d) subjecting at least part of the stream comprising C4 hydrocarbons from both the cracker effluent and the oxygenate conversion effluent to extractive distillation to obtain a stream enriched in unsaturated C4 hydrocarbons and a stream enriched in saturated C4 hydrocarbons.

2. A process according to claim 1, wherein step c) comprises combining at least part of the cracker effluent and at least part of the oxygenate conversion effluent to obtain a combined effluent, and separating from the combined effluent the olefin product stream and the stream comprising C4 hydrocarbons from both the cracker effluent and the oxygenate conversion effluent.

3. A process according to claim 2, wherein butadiene is removed from the stream comprising C4 hydrocarbons from both the cracker effluent and the oxygenate conversion effluent obtained in step c) prior to subjecting the stream comprising C4 hydrocarbons to extractive distillation in step d).

4. A process according to claim 1, wherein step c) comprises:—separately subjecting each of the cracker effluent and the oxygenate conversion effluent to one or more separation steps such that a first stream comprising C4 hydrocarbons and a second stream comprising C4 hydrocarbons are obtained, wherein the first stream comprises C4 hydrocarbons from the cracker effluent and the second stream comprises C4 hydrocarbons from the oxygenate conversion effluent; and
combining the first and the second stream comprising C4 hydrocarbons to obtain the stream comprising C4 hydrocarbons including saturated and unsaturated C4 hydrocarbons from both the cracker effluent and the oxygenate conversion effluent.

5. A process according to claim 4, wherein butadiene is removed from the first stream comprising C4 hydrocarbons prior to combining the first and the second stream comprising C4 hydrocarbons.

6. A process according to claim 3, wherein butadiene is removed by means of extractive distillation.

7. A process according to claim 1 wherein the stream enriched in saturated C4 hydrocarbons obtained in step d) is at least partly recycled to cracking step a).

8. A process according to claim 1 wherein the stream enriched in unsaturated C4 hydrocarbons obtained in step d) is at least partly recycled to oxygenate conversion step b).

9. A process according to claim 1 further comprising:
e) supplying at least part of the stream enriched in unsaturated C4 hydrocarbons obtained in step d) and an alcohol selected from the group consisting of methanol, ethanol and a mixture thereof, to an etherification reaction zone comprising an etherification catalyst and reacting, in the etherification reaction zone, at least part of the isobutene in the stream enriched in unsaturated C4 hydrocarbons with the alcohol to obtain an etherification product stream comprising alkyl tertiary butyl ether;
f) separating the etherification product stream into an alkyl tertiary butyl ether-enriched stream and an isobutene-depleted unsaturated C4 hydrocarbon stream; and
g) recycling at least part of the isobutene-depleted unsaturated C4 hydrocarbon stream and/or at least part of the alkyl tertiary butyl ether, optionally after conversion into tertiary butanol and/or isobutene, to oxygenate conversion step b).

10. A process according to claim 9, wherein at least part of the isobutene-depleted unsaturated C4 hydrocarbon stream is recycled to step b).

11. A process according to claim 1 wherein the molecular sieve-containing catalyst is a zeolite-comprising catalyst.

12. A process according to claim 11, wherein the zeolite-comprising catalyst comprises at least one of ZSM-5, ZSM-11, ZSM-22 and ZSM-23 zeolites.

13. A process according to claim 1 where the oxygenate is methanol, dimethylether, or a mixture thereof.

14. A method according to claim 1 wherein the olefin product stream in step (c) comprises ethylene and at least part of the ethylene is further converted into at least one of polyethylene, mono-ethylene-glycol, ethylbenzene and styrene monomer.

15. A method according to claim 1 wherein the olefin product stream in step (c) comprises propylene and at least part of the propylene is further converted into at least one of polypropylene and propylene oxide.
